(19) United States
(12) Patent Application Publication
(10) Pub. No.: US 2025/0304944 A1
(43) Pub. Date: Oct. 2, 2025

(54) STABILIZED LIQUID ENZYME COMPOSITIONS FOR BREWING

(71) Applicant: Novozymes A/S, Bagsvaerd (DK)

(72) Inventor: Lone Kierstein Nielsen, Kgs. Lyngby (DK)

(73) Assignee: Novozymes A/S, Bagsvaerd (DK)

(21) Appl. No.: 19/234,014

(22) Filed: Jun. 10, 2025

Related U.S. Application Data
(63) Continuation of application No. 17/774,753, filed on May 5, 2022, now Pat. No. 12,359,187.

(30) Foreign Application Priority Data
Nov. 8, 2019 (EP) 19208025.7

(51) Int. Cl. C12N 9/96; C12N 9/26; C12N 9/44

(52) U.S. Cl. CPC C12N 9/96; C12N 9/2411; C12N 9/2457; C12Y 302/01003; C12Y 302/01041

(57) ABSTRACT

The invention provides liquid enzyme compositions which are physically and microbially stable. The compositions are used, for example, in beer brewing processes.

### CROSS-REFERENCE TO RELATED APPLICATIONS

This application is a continuation of U.S. patent application Ser. No. 17/774,753, filed May 5, 2022, which is a 35 U.S.C. 371 national application of PCT/EP2020/081228, filed Nov. 6, 2020, which claims priority or the benefit from European Patent Application No. 19208025.7, filed Nov. 8, 2019. The contents of these applications are fully incorporated herein by reference.

### REFERENCE TO SEQUENCE LISTING

This application contains a Sequence Listing in computer-readable form created on Jun. 10, 2025 as an xml file, 6 bytes in size, and named SQ_ST26.txt., which is incorporated herein by reference.

### FIELD OF THE INVENTION

The present invention relates to liquid enzyme compositions, which are physically and microbially stable. The compositions are used, for example, in beer brewing processes.

### BACKGROUND

Industrial enzymes are used in many different industries, such as household care, food, feed, and biofuels, and are supplied as both solid and liquid products. When liquid enzyme products are shipped across the world, and/or stored in warehouses, it is important that the products are sufficiently stable to maintain specifications, even when they reach the customers a long time after production. Stability includes both enzyme stability, physical stability, and microbial stability.

Microbial stability of liquid enzyme products is traditionally achieved by using preservation agents. Many different preservation agents are known, but since they act by excerting a biocidal effect, there is a desire not to use preservation agents, if possible; in particular in the food industry.

However, the choice of formulation ingredients used to develop such preservative-free and microbially stable formulations is not a simple one, because it will also affect both the enzyme stability and physical stability of the final liquid product, due to (in) compatibility issues.

### SUMMARY OF THE INVENTION

The present invention provides, in a first aspect, a liquid composition, comprising
a) 0.01 to 30% w/w active enzyme protein of one or more carbohydrate degrading enzymes from EC 3.2.1.-,
b) more than 30% w/w of one or more polyols,
c) less than 10% w/w of one or more inorganic salts, and
d) a pH of less than 5;
wherein at least 40% w/w of the polyols are non-sugar polyols, and wherein the composition is substantially free of benzoates, sorbates, and sulfites.

Other aspects and embodiments of the invention are apparent from the description and examples.

Unless otherwise indicated, or if it is apparent from the context that something else is meant, all percentages are percentage by weight (% w/w).

### DETAILED DESCRIPTION

We have found that it is possible to prepare preservative-free liquid enzyme products that maintains microbial stability, while not weakening the enzyme stability or the physical stability, by carefully selecting the ingredients and amounts used in the liquid enzyme composition. Thus, the liquid composition maintains microbial stability while being substantially free of commonly used preservation agents for use in foods, like benzoates, sorbates, and/or sulfites.

Microbial stability is the ability to resist microbial growth. This may be evaluated by inoculating the liquid composition with microorganisms, and measure the subsequent growth of the microorganisms to confirm that they are not proliferating.

Physical stability is the ability to maintain a transparent, preferably clear, composition. This may be evaluated by visual inspection, or by centrifugation. For example, the liquid composition may centrifugated at 1200 G for 10 minutes to determine if a pellet (solid phase) is formed. Alternatively, transparency may be measured as turbidity or haziness, by using a nephelometer to measure NTU to determine light scattering.

Enzymatic stability is the ability to maintain enzymatic activity after storage. This may be determined by measuring the enzymatic activity before and after storage (for example, 8 weeks storage at 25° C.) to determine how much activity is lost. For practical purposes, the residual activity may be determined by comparing the activity of a stored sample and a frozen reference sample, which are analyzed at the same time to eliminate analytical day-to-day variation.

The formulations of the invention were developed for use in a beer brewing process, but are generally applicable also in other processes.

### Definitions

The term “malt” is understood as any malted cereal grain, in particular barley.

The term “mash” is understood as a starch containing slurry comprising crushed barley malt, crushed unmalted grain, other starch containing material, or a combination hereof, steeped in water to make wort.

The term “wort” is understood as the unfermented liquor run-off following extracting the grist during mashing.

The term “beer” is here understood as fermented wort, i.e. an alcoholic beverage brewed from barley malt, optionally adjunct and hops. The term “beer” as used herein is intended to cover at least beer prepared from mashes prepared from unmalted cereals as well as all mashes prepared from malted cereals, and all mashes prepared from a mixture of malted and unmalted cereals. The term “beer” also covers beers prepared with adjuncts, and beers with all possible alcohol contents.

### Liquid Enzyme Composition

The liquid enzyme composition of the invention comprises:
a) 0.01 to 30% w/w active enzyme protein of one or more carbohydrate degrading enzymes from EC 3.2.1.-,
b) more than 30% w/w of one or more polyols,
c) less than 10% w/w of one or more inorganic salts, and
d) a pH of less than 5;
wherein at least 40% w/w of the polyols are non-sugar polyols, and wherein the composition is substantially free of benzoates, sorbates, and sulfites.

The liquid composition maintains physical stability after storage (such as 8 weeks storage at 25° C.), and in a preferred embodiment, the liquid composition is transparent. The liquid composition may be transparent if there is essentially no solid phase after centrifugation at 1200 G for 10 minutes.

As mentioned above, the liquid composition also maintains excellent enzymatic stability. The residual enzymatic activity may be at least 90% after 8 weeks storage at 25° C.

The liquid composition may be an aqueous liquid composition, comprising more than 5% w/w of water; preferably more than 10% w/w of water, more preferably more than 15% w/w of water, and most preferably more than 20% w/w of water.

### Carbohydrate Degrading Enzyme

The carbohydrate degrading enzymes used in the compositions of the invention are catalytic proteins, and the term “active enzyme protein” is defined herein as the amount of catalytic protein(s), which exhibits enzymatic activity. This can be determined using an activity based analytical enzyme assay. In such assays, the enzyme typically catalyzes a reaction generating a colored compound. The amount of the colored compound can be measured and correlated to the concentration of the active enzyme protein. This technique is well-known in the art.

The carbohydrate degrading enzyme(s) are capable of degrading carbohydrates, in particular water-soluble carbohydrates. The carbohydrate degrading enzyme may be one or more enzymes selected from the group consisting of amylase, endoglucanase, xylanase, and pullulanase. In an embodiment, the carbohydrate degrading enzyme is an amylase and/or a pullulanase.

The amylase may be fungal or bacterial; e.g., an alpha-amylase (EC 3.2.1.1) from, e.g., Bacillus, e.g. B. licheniformis or B. amyloliquefaciens; a beta-amylase (EC 3.2.1.2) from, e.g., plant (e.g. soy bean) or from microbial sources (e.g., Bacillus); a fungal alpha-amylase, e.g., from A. oryzae or A. niger, a glucoamylase/amyloglucosidase (EC 3.2.1.3) from, e.g., an Aspergillus or Talaromyces species; or an isoamylase (E.C. 3.2.1.68).

The glucoamylase may have a sequence identity of at least 60%, at least 65%, at least 70%, at least 75%, at least 80%, at least 85%, at least 90%, at least 95%, at least 96%, at least 97%, at least 98%, or at least 99% to the amino acid sequence of the Aspergillus niger G1 or G2 glucoamylase (Boel et al. (1984), EMBO J. 3 (5), p. 1097-1102), the A. awamori glucoamylase disclosed in WO 84/02921, or the A. oryzae glucoamylase (Agric. Biol. Chem. (1991), 55 (4), p. 941-949).

The endoglucanase (E.C. 3.2.1.4) may be derived from a filamentous fungus, such as Aspergillus, Trichoderma, Humicola, or Fusarium.

The xylanase (EC 3.2.1.8 and EC 3.2.1.32) may be derived from Aspergillus aculeatus.

The pullulanase (EC 3.2.1.41) may be derived from Bacillus deramificans (see for example U.S. Pat. No. 5,736,375 or WO 2006/066579).

In a particularly preferred embodiment, the carbohydrate degrading enzyme is a glucoamylase/amyloglucosidase (EC 3.2.1.3) and/or a pullulanase (EC 3.2.1.41).

The enzyme may be a naturally occurring enzyme of bacterial or fungal origin, or it may be a variant derived from one or more naturally occurring enzymes by gene shuffling and/or by substituting, deleting or inserting one or more amino acids. Chemically modified or protein engineered mutants are included.

The liquid composition contains at least one enzyme in an amount of 0.01-30% w/w active enzyme protein; preferably in an amount of 0.05-30% w/w active enzyme protein; more preferably in an amount of 0.1-30% w/w active enzyme protein, and most preferably in an amount of 0.1-25% w/w active enzyme protein.

### Polyols

The polyols (or polyhydric alcohols) as used in the liquid composition of the invention are alcohols with two or more hydroxyl groups. The polyols typically have a molecular weight lower than 500 g/mol.

Polyols include suitable sugar polyols, such as mono- and disaccharides, like glucose, fructose, galactose, sucrose, lactose, maltose, and trehalose.

Polyols also include suitable non-sugars polyols, such as glycerol, ethylene glycol, diethylene glycol, triethylene glycol, propylene glycol, dipropylene glycol, tripropylene glycol, polyethylene glycol (PEG), and sugar alcohols. The polyethylene glycol may have an average molecular weight at or below about 500. Examples of sugar alcohols are sorbitol, mannitol, erythritol, dulcitol, inositol, xylitol and adonitol.

The liquid composition contains more than 30% w/w of one or more polyols, preferably more than 35% w/w of one or more polyols, and most preferably more than 40% w/w of one or more polyols.

At least 40% w/w of the polyols are non-sugar polyols, as described above. Preferably, at least 45% w/w, or at least 50% w/w, of the polyols are non-sugar polyols.

The liquid composition may comprise more than 2% w/w of one or more sugar polyols. In an embodiment, the liquid composition comprises at least 5% w/w, preferably at least 10% w/w, of one or more sugar polyols, as described above.

### Inorganic Salts

Salts are commonly used in liquid enzyme formulations; however, we have observed that when more than 10% w/w salt is used in the liquid formulation of the invention, it is detrimental to the physical stability. Thus, the liquid enzyme composition comprises less than 10% w/w of one or more inorganic salts, preferably less than 5% w/w of one or more inorganic salts.

The inorganic salts may be selected from the group consisting of Na, K, NH₄, Ca, Mg, and Zn salts of mono- or divalent anions. Examples of anions include chloride, sulphate, nitrate, phosphate, formate, and acetate.

Further embodiments of the invention include:

Embodiment 1. A liquid composition, comprising
a) 0.01 to 30% w/w active enzyme protein of one or more carbohydrate degrading enzymes from EC 3.2.1.-,
b) more than 30% w/w of one or more polyols,
c) less than 10% w/w of one or more inorganic salts, and
d) a pH of less than 5;
wherein at least 40% w/w of the polyols are non-sugar polyols, and wherein the composition is substantially free of benzoates, sorbates, and sulfites.

Embodiment 2. The composition of embodiment 1, which comprises 0.05-30% w/w active enzyme protein of one or more carbohydrate degrading enzymes from EC 3.2.1.-.

Embodiment 3. The composition of embodiment 1, which comprises 0.1-30% w/w active enzyme protein of one or more carbohydrate degrading enzymes from EC 3.2.1.-.

Embodiment 4. The composition of embodiment 1, which comprises 0.5-30% w/w active enzyme protein of one or more carbohydrate degrading enzymes from EC 3.2.1.-.

Embodiment 5. The composition of embodiment 1, which comprises 0.1-25% w/w active enzyme protein of one or more carbohydrate degrading enzymes from EC 3.2.1.-.

Embodiment 6. The composition of embodiment 1, which comprises 0.5-25% w/w active enzyme protein of one or more carbohydrate degrading enzymes from EC 3.2.1.-.

Embodiment 7. The composition of any of embodiments 1-6, which comprises more than 35% w/w of polyols.

Embodiment 8. The composition of any of embodiments 1-7, which comprises at least 40% w/w of polyols.

Embodiment 9. The composition of any of embodiments 1-8, which comprises less than 5% w/w of inorganic salts.

Embodiment 10. The composition of any of embodiments 1-9, wherein the pH is less than 4.5.

Embodiment 11. The composition of any of embodiments 1-10, wherein at least 45% w/w of the polyols are non-sugar polyols.

Embodiment 12. The composition of any of embodiments 1-11, wherein at least 50% w/w of the polyols are non-sugar polyols.

Embodiment 13. The composition of any of embodiments 1-12, wherein the carbohydrate degrading enzymes are selected from the group consisting of amylase, endoglucanase, xylanase, and pullulanase.

Embodiment 14. The composition of any of embodiments 1-13, wherein the carbohydrate degrading enzymes are selected from the group consisting of amylase and pullulanase.

Embodiment 15. The composition of any of embodiments 1-14, wherein the carbohydrate degrading enzymes are selected from the group consisting of alpha-amylase, glucoamylase, and pullulanase.

Embodiment 16. The composition of any of embodiments 1-15, wherein the carbohydrate degrading enzymes are selected from the group consisting of glucoamylase and pullulanase.

Embodiment 17. The composition of any of embodiments 1-16, wherein the polyols have two or more hydroxyl groups, and a molecular weight lower than 500 g/mol.

Embodiment 18. The composition of any of embodiments 1-17, wherein the polyols are selected from the group consisting of monosaccharides, disaccharides, and non-sugar polyols.

Embodiment 19. The composition of any of embodiments 1-18, wherein the monosaccharides are selected from the group consisting of glucose, fructose, and galactose.

Embodiment 20. The composition of any of embodiments 1-19, wherein the disaccharides are selected from the group consisting of sucrose, lactose, maltose, and trehalose.

Embodiment 21. The composition of any of embodiments 1-20, wherein the non-sugar polyols are selected from the group consisting of glycerol, ethylene glycol, diethylene glycol, triethylene glycol, propylene glycol, dipropylene glycol, tripropylene glycol, polyethylene glycol (PEG), sorbitol, mannitol, erythritol, dulcitol, inositol, xylitol, and adonitol.

Embodiment 22. The composition of any of embodiments 1-21, which is transparent.

Embodiment 23. The composition of any of embodiments 1-22, wherein there is essentially no solid phase after centrifugation of the composition at 1200 G for 10 mins.

Embodiment 24. The composition of any of embodiments 1-23, which comprises more than 5% w/w of water.

Embodiment 25. The composition of any of embodiments 1-24, which comprises more than 10% w/w of water.

Embodiment 26. The composition of any of embodiments 1-25, which comprises more than 15% w/w of water.

Embodiment 27. The composition of any of embodiments 1-26, which comprises more than 20% w/w of water.

Embodiment 28. The composition of any of embodiments 1-27, which comprises more than 2% w/w of one or more sugar polyols.

Embodiment 29. The composition of any of embodiments 1-28, which comprises more than 5% w/w of one or more sugar polyols.

Embodiment 30. The composition of any of embodiments 1-29, which comprises more than 10% w/w of one or more sugar polyols.

Embodiment 31. The composition of any of embodiments 1-30, wherein the % refractive index is higher than 45.

Embodiment 32. A method for producing a brewer's wort, comprising adding the liquid composition of any of embodiments 1-31 to a mash.

The present invention is further described by the following examples which should not be construed as limiting the scope of the invention.

### EXAMPLES

Chemicals were commercial products of at least reagent grade.

Glucoamylase1 (glucan 1,4-alpha-glucosidase, EC 3.2.1.3, also referred to as amyloglucosidase) has the amino acid sequence shown in SEQ ID NO: 1.

Glucoamylase2 has the amino acid sequence shown in SEQ ID NO: 2.

Pullulanase (alpha-dextrin endo-1,6-alpha-glucosidase, EC 3.2.1.41) has the amino acid sequence shown in SEQ ID NO: 3.

Glucoamylase activity is measured as specified in the analytical method “AGU, Amyloglucosidase determination” from Novozymes. Briefly, amyloglucosidase hydrolyzes maltose to form α-D-glucose. After incubation, the reaction is stopped with NaOH. Glucose is phosphorylated by ATP in a reaction catalyzed by hexokinase. The glucose-6-phosphate which is formed is oxidized to 6-phosphogluconate by glucose-6-phosphate dehydrogenase. In this same reaction, an equimolar amount of NAD+ is reduced to NADH with a resultant increase in absorbance at 340 nm.

Pullulanase activity is measured as specified in the analytical method “PUN (J), Pullulanase determination” from Novozymes. Briefly, pullulanase cleaves the substrate 4,6-O-benzylidene-4-nitrophenyl-6-α-D-maltotriosyl-maltotriose. α-glucosidase removes glucose units from the non-reducing end of the formed 4-nitrophenyl-β-maltotrioside, and β-glucosidase releases 4-nitrophenyl, which-after a pH change from pH 5.0 to a basic pH>9.0—can be monitored at 405 nm in a Konelab analyzer.

% Refractive Index is measured using an ATAGO PAL-alpha pocket refractometer. % RI is the corresponding % sucrose concentration (Brix).

### Example 1

### Stable Liquid Glucoamylase Composition

A liquid glucoamylase (amyloglucosidase) formulation, with the composition and characteristics as shown in Table 1, was prepared and tested for physical stability, enzymatic stability, and microbial stability, as shown in Tables 2-5.

TABLE 1
Liquid enzyme formulation.

| Parameter | Value |
|---|---|
| Glucoamylase1 amount | 20% w/w active enzyme protein |
| Glycerol | 50% w/w |
| pH | 4.3 |
| % Refractive Index | 58 |
| Visual appearance (t = 0) | Clear |

TABLE 2
Physical stability of the liquid enzyme
formulation after incubation.

| Physical stability | Visual inspection |
|---|---|
| 4 weeks at 10° C. | Clear |
| 4 weeks at 25° C. | Clear |
| 13 weeks at 10° C. | Clear |
| 13 weeks at 25° C. | Clear |

TABLE 3
Residual enzymatic activitiy after
incubation for 4 and 13 weeks.

| Incubation | | |
|---|---|---|
| temperature | 4 weeks | 13 weeks |
| −18° C. | 100% | 100% |
| 10° C. | 100% | 101% |
| 25° C. | 100% | 98% |
| 40° C. | 99% | 95% |

### Microbial Stability

The formulation was shown to be microbially robust towards bacteria, lactobacilli as well as yeast and mold. This was done by spiking/challenging the formulation with the microorganisms in Table 4. Each of the three bottles were inoculated to a total of 1×10⁵ CFU/ml of the test microorganisms.

TABLE 4
Test microorganisms used in bottles 1-3.

| Bottle | Test organism |
|---|---|
| 1 | Escherichia coli |
| | Pseudomonas aeruginosa |
| | Salmonella havana |
| | Acinetobacter spp. |
| | Staphylococcus aureus |
| | Staphylococcus xylosus |
| | Enterococcus faecium |
| 2 | Lactobacillus buchneri |
| | Lactobacillus para paracasei |
| 3 | Aspergillus niger |
| | Candida parapsilosis |
| | Candida famata |

The bottles were analyzed for CFU/ml before inoculation (blind) and after 1, 2, 3, and 5 weeks of incubation at 20-25° C. CFU (colony forming units) per ml was measured using standard microbiological methods.

TABLE 5
Microbial stability of the liquid enzyme formulation, measured as CFU/ml.

| Bottle | Blind | Added | 1 week | 2 weeks | 3 weeks | 5 weeks |
|---|---|---|---|---|---|---|
| 1 | 1.0 × 10² | 1.0 × 10⁵ | 2.4 × 10³ | 1.0 × 10² | 1.0 × 10² | 1.0 × 10² |
| 2 | 1.0 × 10² | 1.0 × 10⁵ | 1.0 × 10² | 1.0 × 10² | 1.0 × 10² | 1.0 × 10² |
| 3 | 1.0 × 10² | 1.0 × 10⁵ | 2.0 × 10⁴ | 3.5 × 10³ | 5.0 × 10³ | 1.1 × 10³ |

The data in Tables 2 and 3 show that the liquid enzyme composition of Table 1 maintains physical and enzymatic stability even after incubation for up to 13 weeks at 25° C.

The data in Table 5 show that the liquid enzyme composition of Table 1 is microbially robust towards all microorganisms in bottles 1, 2 and 3 based on a success criterea of >1 log reduction of all added test microorganisms in bottles 1, 2 and 3 after incubation for less than 4 weeks.

### Example 2

### Stable Liquid Glucoamylase/Pullulanase Composition

A liquid glucoamylase/pullulanase formulation, with the composition and characteristics as shown in Table 6, was prepared and tested for physical stability, enzymatic stability, and microbial stability, as shown in Tables 7-9.

TABLE 6
Liquid enzyme formulation.

| Parameter | Value |
|---|---|
| Glucoamylase2 amount | 25% w/w active enzyme protein |
| Pullulanase amount | 0.3% w/w active enzyme protein |
| Glycerol | 20% w/w |
| Glucose | 20% w/w |
| pH | 4.0 |
| % Refractive Index | >50 |
| Visual appearance (t = 0) | Clear |

TABLE 7
Physical stability of the liquid enzyme
formulation after incubation.

| Physical stability | Visual inspection |
|---|---|
| 8 weeks at 10° C. | Clear |
| 8 weeks at 25° C. | Transparent |

TABLE 8
Residual enzymatic activitiy after
incubation for 8 weeks.

| Incubation | Residual activity after 8 weeks | |
|---|---|---|
| temperature | Glucoamylase | Pullulanase |
| −18° C. | 100% | 100% |
| 25° C. | 96% | 99% |

### Microbial Stability

The formulation was shown to be microbially robust towards bacteria, lactobacilli as well as yeast and mold. This was done by spiking/challenging the formulation with the microorganisms in Table 4 of Example 1. Each of the three bottles were inoculated to a total of 1×10⁵ CFU/ml of the test microorganisms.

The bottles were analyzed for CFU/ml before inoculation (blind) and after 1, 2, 3, and 4 weeks of incubation at 20-25° C. CFU (colony forming units) per ml was measured using standard microbiological methods.

TABLE 9
Microbial stability of the liquid enzyme formulation, measured as CFU/ml.

| Bottle | Blind | Added | 1 week | 2 weeks | 3 weeks | 4 weeks |
|---|---|---|---|---|---|---|
| 1 | 8.0 × 10² | 1.0 × 10⁵ | 1.0 × 10² | 1.0 × 10² | 1.0 × 10² | 4.0 × 10² |
| 2 | 1.0 × 10² | 1.0 × 10⁵ | 1.0 × 10² | 1.0 × 10² | 1.0 × 10² | 1.0 × 10² |
| 3 | 1.1 × 10³ | 1.0 × 10⁵ | 1.0 × 10² | 1.0 × 10² | 1.0 × 10² | 1.1 × 10³ |

The data in Tables 7 and 8 show that the liquid enzyme composition of Table 6 maintains physical and enzymatic stability even after incubation for 8 weeks at 25° C.

The data in Table 9 show that the liquid enzyme composition of Table 6 is microbially robust towards all microorganisms in bottles 1, 2 and 3 based on a success criterea of >1 log reduction of all added test microorganisms in bottles 1, 2 and 3 after incubation for less than 4 weeks.

1. A liquid enzyme composition, comprising:
a) 0.01 to 30% w/w active enzyme protein of pullulanase,
b) more than 30% w/w of one or more polyols,
c) less than 10% w/w of one or more inorganic salts, and
d) a pH of less than 5;
wherein at least 40% w/w of the polyols are non-sugar polyols, and
wherein the composition is substantially free of benzoates, sorbates, and sulfites.

2. The composition of claim 1, which is transparent.

3. The composition of claim 1, wherein there is essentially no solid phase after centrifugation of the composition at 1200 G for 10 mins.

4. (canceled)

5. The composition of claim 1, which further comprises a glucoamylase.

6. The composition of claim 1, wherein the polyols have two or more hydroxyl groups, and a molecular weight lower than 500 g/mol.

7. The composition of claim 1, wherein the polyols are selected from the group consisting of monosaccharides, disaccharides, and non-sugar polyols.

8. The composition of claim 1, wherein the non-sugar polyols are selected from the group consisting of glycerol, ethylene glycol, diethylene glycol, triethylene glycol, propylene glycol, dipropylene glycol, tripropylene glycol, polyethylene glycol (PEG), sorbitol, mannitol, erythritol, dulcitol, inositol, xylitol, and adonitol.

9. The composition of claim 1, wherein the inorganic salt(s) are selected from the group consisting of Na, K, NH₄, Ca, Mg, and Zn salts of mono- or divalent anions.

10. The composition of claim 1, wherein the mono- or divalent anions are selected from the group consisting of chloride, sulphate, nitrate, phosphate, formate, and acetate.

11. The composition of claim 1, which comprises more than 35% w/w of one or more polyols.

12. The composition of claim 1, which comprises less than 5% w/w of one or more inorganic salts.

13. The composition of claim 1, which comprises a pH of less than 4.5.

14. The composition of claim 1, which comprises at least 10% w/w of water.

15. The composition of claim 1, which comprises more than 2% w/w of one or more sugar polyols.

16. A method for producing a brewer's wort, the method comprising adding the liquid composition of claim 1 to a mash.

17. The composition of claim 1, which comprises at least 20% w/w of water.

18. The composition of claim 1, which comprises more than 5% w/w of one or more sugar polyols.

19. The composition of claim 1, which comprises more than 10% w/w of one or more sugar polyols.

20. The composition of claim 1, wherein at least 50% w/w of the polyols are non-sugar polyols.

21. The composition of claim 1, wherein the % refractive index is higher than 45.


SEQUENCE LISTING (3 sequences)

SEQ ID NO: 1 - protein, 622 aa, Aspergillus niger
NVISKRATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEEVNGSSFFTIAVQHRALVEGSAFATAVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 2 - protein, 591 aa, Talaromyces emersonii
ATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 3 - protein, 828 aa, synthetic construct
  REGION 1..828: note = Pullulanase derived from Bacillus deramificans
DSTSTEVIVHYHRFDSNYANWDLWMWPYQPVNGNGAAYEFSGKDDFGVKADVQVPGDDTQVGLIVRTNDWSQKNTSDDLHIDLTKGHEIWIVQGDPNIYYNLSDAQAAATPKVSNAYLDNEKTVLAKLTNPMTLSDGSSGFTVTDKTTGEQIPVTAATNANSASSSEQTDLVQLTLASAPDVSHTIQVGAAGYEAVNLIPRNVLDSSQYYYSGDDLGNTYTHKATTFKVWAPTSTQVNVLLYNSATGSVTKTVPMTASGHGVWEATVNQNLENWYYMYEVTGQGSTRTAVDPYATAIAPNGTRGMIVDLAKTDPAGWNSDKHITPKNIEDEVIYEMDVRDFSIDPNSGMKNKGKYLALTEKGTKGPDGVKTGIDSLKQLGITHVQLMPVFAFASVDETDPTQDNWGYDPRNYDVPEGQYATNANGTARIKEFKEMVLSLHREHIGVNMDVVYNHTFATQISDFDKIVPEYYYRTDDAGNYTNGSGTGNEIASERPMVQKFIIDSLKYWVNEYHIDGFRFDLMALLGKDTMSKAASELHAINPGIALYGEPWTGGTSALPEDQLLTKGAQKGMGVAVFNDNLRNALDGNVFDSSAQGFATGATGLTDAIKNGVEGSINDFTSSPGETINYVTSHDNYTLWDKIALSNPNDSEADRIKMDELAQAVVMTSQGVPFMQGGEEMLRTKGGNDNSYNAGDTVNEFDWSRKAQYPDVFNYYSGLIHLRLDHPAFRMTTANEINSHLQFLNSPENTVAYELTDHVNKDKWGNIIVVYNPNKTAATINLPSGKWAINATSGKVGESTLGQAEGSVQVPGISMMILHQEVSPDHGKK